(19) United States
(12) Patent Application Publication
(10) Pub. No.: US 2020/0215305 A1
(43) Pub. Date: Jul. 9, 2020

(54) UNIVERSAL CATHETER TIP AND METHODS OF MANUFACTURING

(71) Applicant: SANFORD HEALTH, Sioux Falls, SD (US)

(72) Inventor: Patrick W. KELLY, Sioux Falls, SD (US)

(21) Appl. No.: 16/641,925

(22) PCT Filed: Aug. 30, 2018
(86) PCT No.: PCT/US2018/048768

§ 371 Date: Feb. 25, 2020

Related U.S. Application Data
(60) Provisional application No. 62/552,168, filed on Aug. 30, 2017.

(51) Int. Cl. A61M 25/00; A61F 2/07; A61B 17/12; A61B 17/3205; A61F 2/95

(52) U.S. Cl. CPC A61M 25/0074; A61M 25/001; A61F 2/07; A61B 2017/00292; A61B 17/32056; A61F 2/95; A61M 2025/0079; A61B 17/12022

(57) ABSTRACT

The present disclosure provides a catheter tip (100) including a flexible housing (102) defining a cavity (104) extending between an expandable opening (106) arranged at a first end (108) of the catheter tip and a second end (110) of the catheter tip. The catheter tip further includes a plurality of pivotable arms (112) coupled to and extending longitudinally along the flexible housing. The plurality of pivotable arms are arranged to taper inwardly at the first end of the catheter tip toward a longitudinal axis of the flexible housing. The expandable opening is configured to increase in diameter from a first position to a second position in response to application of an external force to the second end of the flexible housing.

### RELATED APPLICATIONS

This application claims the benefit of priority to U.S. Provisional Application No. 62/552,168 entitled “Universal Catheter Tip and Methods of Manufacturing,” filed on Aug. 30, 2017, the contents of which are hereby incorporated by reference in its entirety.

### BACKGROUND THE INVENTION

The current state of the art for devices that are advanced via guide wires to various arterial configurations requires a separate device for each diameter of guide wire. For example, a larger guide wire having a diameter of 0.9562 mm may be used for certain aspects of a given procedure, while a smaller guide wire having a diameter of 0.254 mm may be used for different aspects of the same procedure. Using current devices, a different catheter having a different size tip would need to be used for each guide wire size. For example, the 0.9652 mm diameter tip cannot be used on the 0.254 mm guide wire, because there would be a ridge created that would catch on lesions that may allow blood to pass through the resulting gap and into the catheter. As a result, the various sized guide wires require a large inventory of devices that may be expensive and difficult to maintain.

### SUMMARY OF THE INVENTION

The present disclosure provides a catheter tip that may be advantageously adjustable for use with guide wires of varying diameters. Such catheter tips may have a length ranging from roughly 4 cm to 60 cm and may have a diameter ranging from 4 French to 25 French depending on the particular device being delivered in vivo. The French size of the catheter refers to the outer diameter that is also termed the catheter's “crossing profile.” Reducing the crossing profile may allow the catheter to cross narrow lesions and to enter smaller blood vessels. The material of the catheter tip may be flexible, kink resistant, atraumatic, and torquable. The material of the catheter tip may be silicone, polyurethane, nylon, or Pebax among other materials. Catheters and tips of the present disclosure may be lined by materials that are hemocompatible, lubricious, non-thrombogenic, and/or sometimes radiopaque. The inner liner materials may be polytetrafluoroethylene (PTFE), polyimide, or high density polyethylene, for example. The mechanical properties of such a catheter tip may include high burst pressure, high tensile strength and compression resistance, high modulus of elasticity and low coefficient of friction. Forming techniques for catheter tips of the present disclosures can include coated extrusions, co-extrusions, coil-reinforced or braid-reinforced composites that may further include an inner lubricious layer, reinforcement layer and an outer jacket layer. The coil- or braid-reinforced metal layers may provide tensile strength, while the polymer layer may provide lubricity and flexibility. The catheter tips are bonded to the catheter in such a way so as to minimize risk of separation from one another during the procedure, because a tip that separates from the catheter can create a situation where blood flow is blocked to a major organ.

Thus, in a first aspect, the present disclosure provides a catheter tip that includes (a) a flexible housing defining a cavity extending between an expandable opening arranged at a first end of the catheter tip and a second end of the catheter tip, and (b) a plurality of pivotable arms coupled to and extending longitudinally along the flexible housing, wherein the plurality of pivotable arms are arranged to taper inwardly at the first end of the catheter tip toward a longitudinal axis of the flexible housing, where the expandable opening is configured to increase in diameter from a first position to a second position in response to application of an external force to the second end of the flexible housing.

In a second aspect, the present disclosure provides a method of forming a catheter tip that includes: (a) forming a plurality of pivotable arms in a linear configuration such that a longitudinal axis of each of the plurality of pivotable arms are in the same plane, and (b) coupling a first edge of a first pivotable arm of the plurality of pivotable arms to a second edge of a second pivotable arm of the plurality of pivotable arms to form a tubular configuration of the plurality of pivotable arms in which the plurality of pivotable arms define a cavity extending between an expandable opening arranged at a first end of the catheter tip and a second end of the catheter tip.

In a third aspect, the present disclosure provides a method that includes: (a) applying a force to the second end of the catheter tip according to the first aspect thereby increasing a diameter of the expandable opening at the first end of the catheter tip, (b) positioning a guide wire through the expandable opening, (c) removing the force from the second end of the catheter tip thereby decreasing the diameter of the expandable opening, and (d) advancing the catheter tip along the guide wire to a target lumen.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A is a side cross-section view of an example catheter tip, according to an example embodiment.

FIG. 1B is a detail view of a pivotable arm embedded in the flexible housing in an unflexed position and in a flexed position shown in dashed lines, according to the example of FIG. 1.

FIG. 2 is a front view of a catheter tip, according to the example of FIG. 1.

FIG. 3 is a side view of an example catheter tip prior to assembly, according to an example embodiment.

FIG. 4 is a side view of another example catheter tip prior to assembly, according to an example embodiment.

FIG. 5 is a rear view of an example catheter tip prior to assembly, according to an example embodiment.

FIG. 6 is a front view of an example catheter tip after assembly, according to an example embodiment.

FIG. 7 is a flow chart depicting functions that can be carried out in accordance with example embodiments of the disclosed methods.

FIG. 8 is another flow chart depicting functions that can be carried out in accordance with example embodiments of the disclosed methods.

### DETAILED DESCRIPTION OF THE INVENTION

The description of the different advantageous arrangements are presented for purposes of illustration and description, and are intended to be exhaustive or limited to the examples in the form disclosed. Many modifications and variations will be apparent to those of ordinary skill in the art. Further, different examples may provide different advantages as compared to other advantageous examples. The example or examples selected are chosen and described in order to best explain the principles of the examples, the practical application, and to enable others of ordinary skill in the art to understand the disclosure for various examples with various modifications as are suited to the particular use contemplated.

As used herein, with respect to measurements, “about” means+/−5%.

As used herein, “coupled” means associated directly, as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” denotes existing characteristics of an apparatus, element, and method which enable the apparatus, element, and method to perform the specified function without further modification. For purposes of this disclosure, an apparatus, element, and method described as being “configured to” perform a particular function may additionally or alternatively be described as being “adapted to” and/or as being “operative to” perform that function.

As used herein, the “flexible housing” may be made of any material capable of being compressed and returning to the same uncompressed configuration, including, but not limited to, a polymer material, such as PLGA (poly-pactic-co-glycolic acid), PCL (poly-caprolactone) or PMMA (poly-methyl-methacrylate), rubber, or silicone.

As used herein, the “pivotable arms” may include a material having a stiffness greater than that of the flexible housing. The pivotable arms may be made of a rigid polymer or of a shape memory metal such as nitinol, as some examples.

As used herein, a “stent graft” is a tubular, radially-expandable device that includes a fluid-tight fabric supported by a stent that may be used to bridge aneurysmal arteries. As such, the term stent graft may be used herein to include bridging stent grafts. Such stent grafts and methods for their deployment and use are known to those of skill in the art. For example, vascular sheaths can be introduced into the patient's arteries, through which items, including but not limited to, guide wires, catheters and, eventually, the stent graft, are passed.

As used herein, “stent” is typically a cylindrical frame and means any device or structure that adds rigidity, expansion force, or support to a prosthesis, while “stent graft” refers to a prosthesis comprising a stent and a graft material associated therewith that forms a fluid-tight or blood-tight lumen through at least a portion of the stent graft's length. A “graft” is a cylindrical liner that may be disposed on the stent's interior, exterior or both. A wide variety of attachment mechanisms are available to join the stent and graft together, including but not limited to, sutures, adhesive bonding, heat welding, and ultrasonic welding.

The stent can be made of any suitable material, including but not limited to biocompatible metals, implantable quality stainless steel wires, nickel and titanium alloys, and biocompatible plastics. The stents can either have material properties necessary to exhibit either self-expanding or balloon-expanding characteristics.

Any suitable fluid tight, or blood tight, graft material can be used. In a preferred embodiment, the graft material is a biocompatible fabric, including but not limited to woven or knitted polyester, such as poly(ethylene terephthalate), polylactide, polyglycolide and copolymers thereof; fluorinated polymers, such as PTFE, expanded PTFE and poly(vinylidene fluoride); polysiloxanes, including polydimethyl siloxane; and polyurethanes, including polyetherurethanes, polyurethane ureas, polyetherurethane ureas, polyurethanes containing carbonate linkages and polyurethanes containing siloxane segments. Materials that are not inherently biocompatible may be subjected to surface modifications in order to render the materials biocompatible. Examples of surface modifications include graft polymerization of biocompatible polymers from the material surface, coating of the surface with a crosslinked biocompatible polymer, chemical modification with biocompatible functional groups, and immobilization of a compatibilizing agent such as heparin or other substances. The graft material may also include extracellular matrix materials.

As used herein, a “catheter” is an apparatus configured to be connected to a deployment mechanism and to house a medical device that can be delivered over a guide wire. The catheter may include a lumen to receive a guide wire for over-the-wire guidance and may be used for delivering a stent graft or other implantable device to a target lumen. A catheter can have braided metal strands within the catheter wall to increase structural integrity. The structural elements of the catheter tip can be bonded or laser welded to the braided strands of the catheter to improve the performance characteristics of the catheter tip.

As used herein, a “guide wire” is an elongated cable comprised of various biocompatible materials that may include metals and polymers. Guide wires may be used for selecting target lumens and guiding catheters to target deployment locations. Guide wires are typically defined as wires used independently of other devices that do not come as part of an assembly.

As used herein, “lumen” refers to a passage within an arterial structure, such as the pulmonary arteries, or the passage within the tubular housings or catheters through which the guide wire may be disposed.

As used herein, “French” refers to a unit of measurement for a catheter. A round catheter of 1 French has an external diameter of ⅓ mm, and therefore the diameter of a round catheter in millimeters can be determined by dividing the French size by 3.

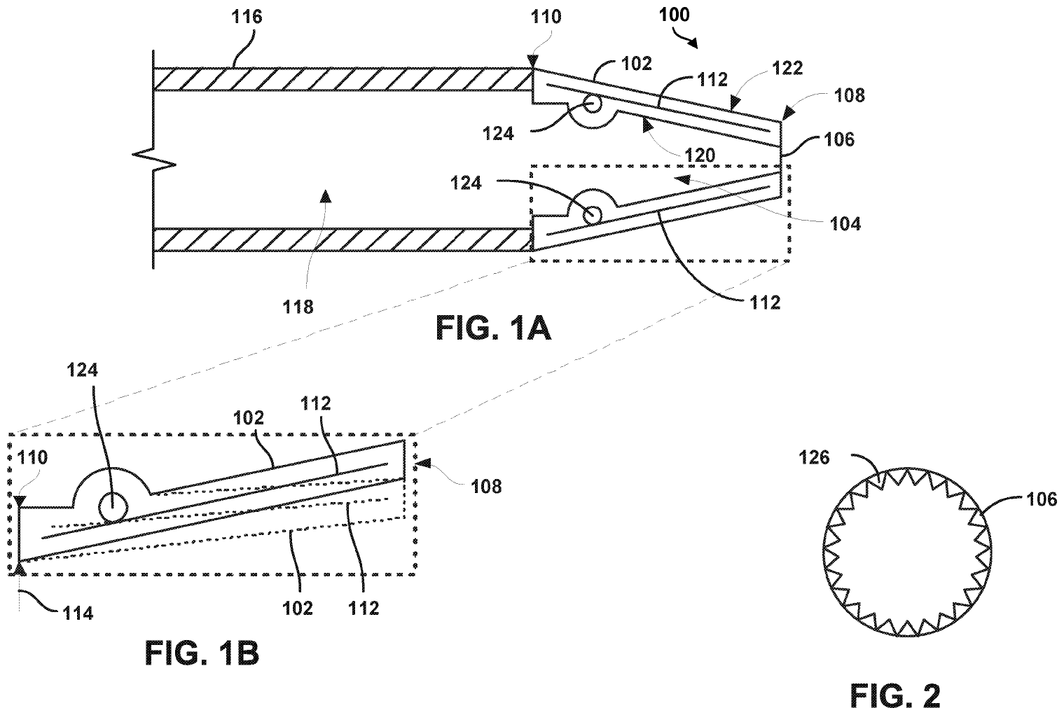

With reference to the Figures, FIG. 1A illustrates an example catheter tip 100. As shown in FIG. 1A, the catheter tip 100 may include a flexible housing 102 defining a cavity 104 extending between an expandable opening 106 arranged at a first end 108 of the catheter tip 100 and a second end 110 of the catheter tip 100. The catheter tip 100 may also include a plurality of pivotable arms 112 coupled to and extending longitudinally along the flexible housing 102. The plurality of pivotable arms 112 may be arranged to taper inwardly at the first end 108 of the catheter tip 100 toward a longitudinal axis of the flexible housing 102.

The flexible housing 102 and/or the expandable opening 106 may be made of any material capable of being stretched and returning to the same unstretched configuration, including, but not limited to, a polymer material such as PLGA (poly-pactic-co-glycolic acid), PCL (poly-caprolactone) or PMMA (poly-methyl-methacrylate), rubber, or silicone. As such, and as shown in FIG. 1B, the expandable opening 106 may be configured to increase in diameter from a first position to a second position in response to application of an external force 114 to the second end 110 of the catheter tip 100. The expandable opening 106 may have a diameter in the first position ranging from about 0.2032 mm to about 0.3556 mm, and the expandable opening 106 may have a diameter in the second position ranging from about 0.2286 mm to about 1.016 mm. The catheter tip 100 may have a tapered transition from the first end 108 to the second end 110, and the cavity 104 at the second end 110 has a greater diameter than at the first end 108. In one particular example, the catheter tip 100 is cone-shaped when the expandable opening 106 is in the first position. Such a configuration may be beneficial to guide the catheter tip 100 through various lumens to a target position. In another example, the catheter tip 100 is bullet-shaped.

In one embodiment, the catheter tip may further include a catheter 116 coupled to the catheter tip 100. In such an example, the catheter tip 100 may further include a guide wire-deployable device positioned within a lumen 118 of the catheter 116. The guide wire-deployable device includes one of an occluder, a pacemaker lead, a snare or a stent graft.

The plurality of pivotable arms 112 may be embedded in the flexible housing 102, coupled to an interior 120 of the flexible housing 102, or coupled to an exterior 122 of the flexible housing 102. In one particular example, the flexible housing 102 is heat shrunk over the plurality of pivotable arms 112. In another embodiment, the catheter tip 100 may further include at least one frame 124 arranged between the plurality of pivotable arms 112 and the cavity 104 and further arranged between a midpoint of each of the plurality of arms 112 and the second end 110 of the catheter tip 100. In such an example, the at least one frame 124 is configured as a pivot point for one or more of the plurality of pivotable arms 112. In such an embodiment, the at least one frame 124 may comprise a ring, a plurality of segments arranged to form a discontinuous ring, a plurality of ball bearings, and/or a plurality of shafts.

In one embodiment, each of the plurality of pivotable arms 112 has a stiffness greater than a stiffness of the flexible housing 102. As such, the plurality of pivotable arms 112 may include a material that is different than the material of the flexible housing 102. The plurality of pivotable arms 112 may be made of any suitable rigid material, such as a rigid polymer, a sheet metal, or a shape memory metal, such as nitinol, as some examples. In one example, each of the plurality of pivotable arms 112 tapers to a point at a first end 108 of the catheter tip 100. In another example, the plurality of pivotable arms 112 includes at least three pivotable arms.

In another embodiment, as shown in FIG. 2, the expandable opening 106 includes a plurality of ridges or teeth 126 configured to grip a guide wire when the expandable opening 106 is in the first position. The ridges or teeth 126 may comprise a different material than the rest of the expandable opening 106. For example, the ridges or teeth 126 may comprise a biocompatible metal. In another example, the ridges or teeth 126 may be the same material as the expandable opening 106.

The expandable opening 106 may take a variety of forms. In one example, the expandable opening 106 may comprise a single ring. In such an example, the expandable opening 106 may be a part of the flexible housing 102 or be made from the same material as the flexible housing 102. In another example, the expandable opening 106 may be a single ring that is permanently coupled to the flexible housing 102. In another example, the expandable opening 106 may comprise a discontinuous ring defined by the plurality of pivotable arms 112 at the first end 108 of the catheter tip 100. As such, the expandable opening 106 may be a meeting of the plurality of pivotable arms 112 in a first position that may be opened up to a larger diameter in a second position.

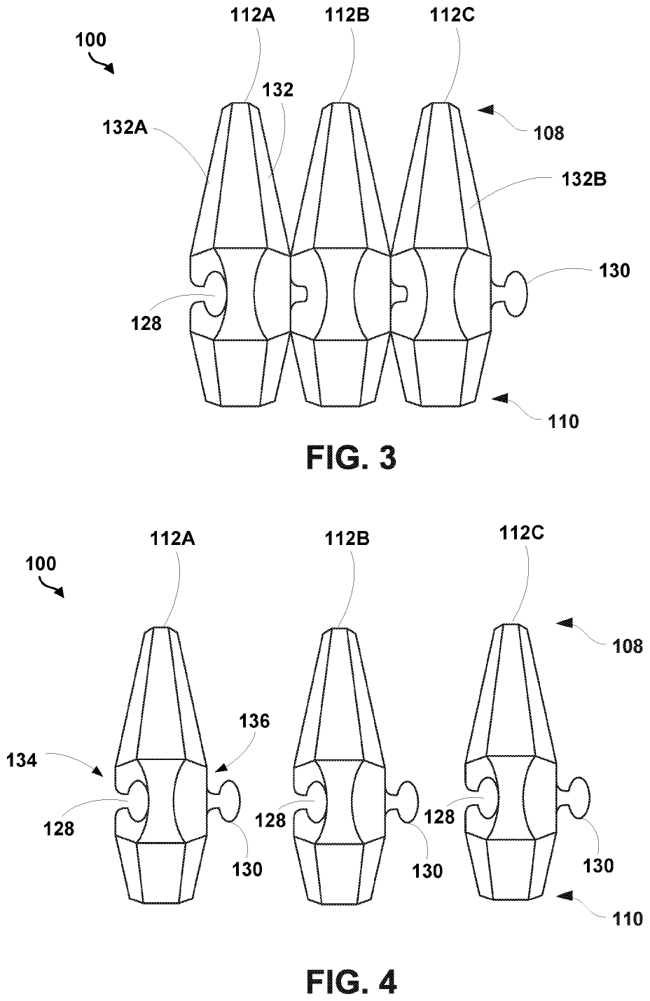

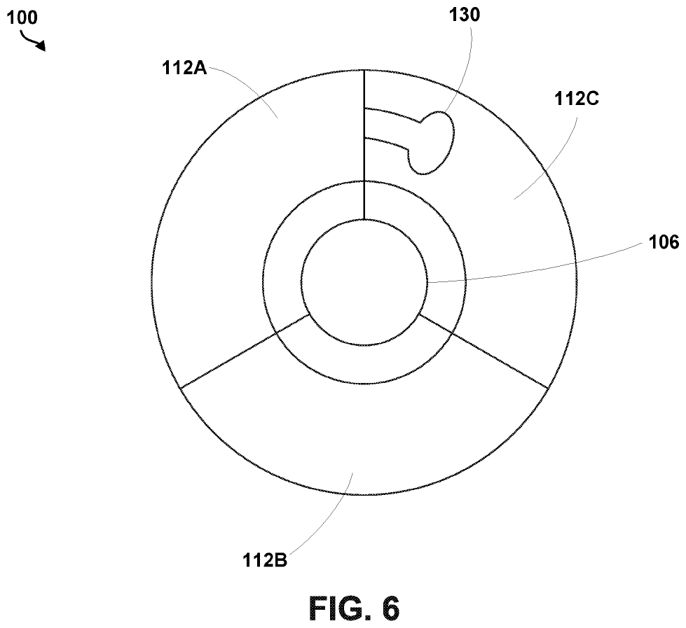

In one embodiment, as shown in FIGS. 3-6, the plurality of pivotable arms 112 are configured to transition from a linear configuration in which a longitudinal axis of each of the plurality of pivotable arms 112 are in the same plane (shown in FIGS. 3-5) to a tubular configuration (shown in FIG. 6) in which the longitudinal axis of each of the plurality of pivotable arms 112 intersect at a center of the expandable opening 106 to thereby form the cavity 104. In such an example, a first pivotable arm 112A of the plurality of pivotable arms 112 may include a locking component 128, a second pivotable arm 112B of the plurality of pivotable arms 112 is coupled to the first pivotable arm 112A, and a third pivotable arm 112C of the plurality of pivotable arms 112 may include a key component 130 configured to be received by the locking component 128 of the first pivotable arm 112A to thereby form the tubular configuration. For example, the locking component 128 may be an opening sized and shaped to reciprocally engage with a key component 130 that has the form of a projection. In another example, as shown in FIG. 4, each of the plurality of pivotable arms 112A-112C includes a locking component 128 and a key component 130, and the key component 130 of each of the plurality of pivotable arms 112 is configured to be received by the locking component 128 of an adjacent pivotable arm 112 of each of the plurality of pivotable arms 112 to thereby form the tubular configuration.

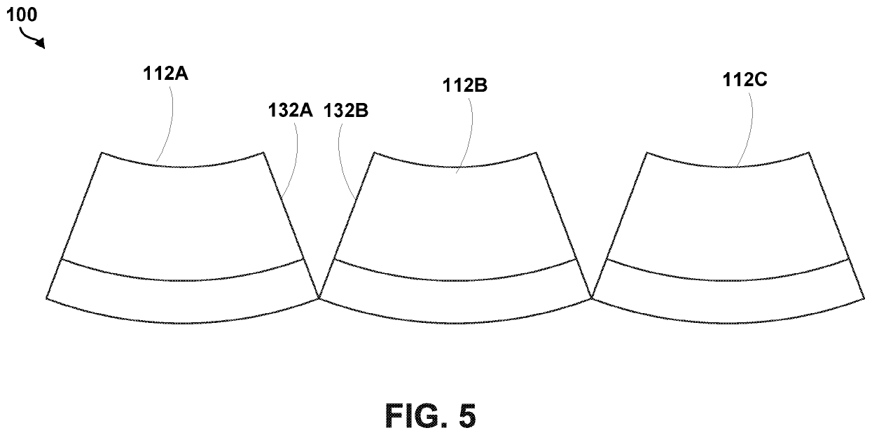

In such examples, each of the plurality of pivotable arms 112 may include a beveled edge 132 complementary to a beveled edge 132 of an adjacent pivotable arm of each of the plurality of pivotable arms 112 to thereby form the tubular configuration. In one particular example, there are three pivotable arms 112 such that the beveled edges 132 have an exterior angle of approximately 120 degrees, as shown in the rear view of the plurality of pivotable arms 112 shown in FIG. 5. Other numbers of pivotable arms 112 are possible as well.

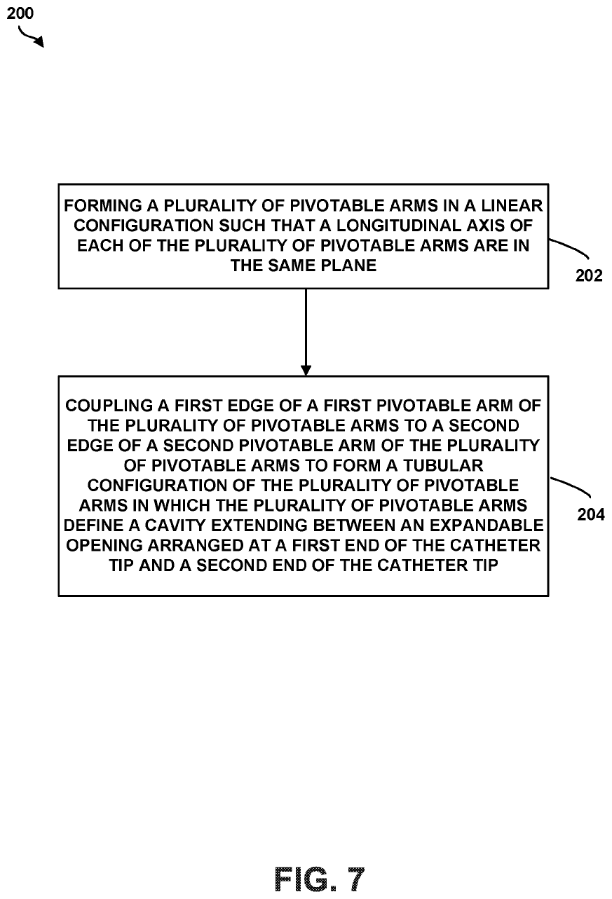

FIG. 7 is a simplified flow chart illustrating a method 200 of forming a catheter tip, such as the catheter tip described above and as shown in FIGS. 1-6, according to an exemplary embodiment. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 202, the method 200 includes forming a plurality of pivotable arms 112 in a linear configuration such that a longitudinal axis of each of the plurality of pivotable arms 112 are in the same plane. At block 204, the method 200 includes coupling a first edge 132A of a first pivotable arm 112A of the plurality of pivotable arms 112 to a second edge 132B of a second pivotable arm 112C of the plurality of pivotable arms 112 to form a tubular configuration of the plurality of pivotable arms 112 in which the plurality of pivotable arms 112 define a cavity 104 extending between an expandable opening 106 arranged at a first end 108 of the catheter tip 100 and a second end 110 of the catheter tip 100.

In one embodiment, a first side 134 of the first pivotable arm 112A of the plurality of pivotable arms 112 may include a locking component 128, and a second side 136 of the second pivotable arm 112B of the plurality of pivotable arms 112 may include a key component 130 configured to receive the locking component 128 of the first pivotable arm 112A to thereby form the tubular configuration. In another example, as shown in FIG. 4, each of the plurality of pivotable arms 112 includes a locking component 128 and a key component 130, and the key component 130 of each of the plurality of pivotable arms 112 is configured to be received by the locking component 128 of an adjacent pivotable arm of each of the plurality of pivotable arms 112 to thereby form the tubular configuration. In one particular example, the plurality of pivotable arms 112 includes at least three pivotable arms. Forming the plurality of pivotable arms 112 in the linear configuration comprises molding the plurality of pivotable arms 112 from a plastic material, or stamping the plurality of pivotable arms from a metallic material, as examples.

In one example, the method 200 may further include positioning a flexible housing 102 over the tubular configuration of the plurality of pivotable arms 112, and applying heat to the flexible housing 102 to thereby secure the flexible housing 102 to the plurality of pivotable arms 112.

As such, the disclosed method 200 of forming a catheter tip 100 provides a method of micro-molding a three dimension structure initially in two dimensions in a chain type configuration, but when separated at every third joint and folded/wrapped around its longitudinal axis forms a three-dimensional cone-shaped catheter tip 100 that can be bonded such to allow movement at the first end 108 of the catheter tip 100 when an external force 114 is applied at or near the second end 110 of the catheter tip 100. The separation of the first end 108 of the catheter tip 100 is about an internally molded frame 124 (e.g., a fulcrum/pivot point) that gives support to the second end 110 of the catheter tip 100.

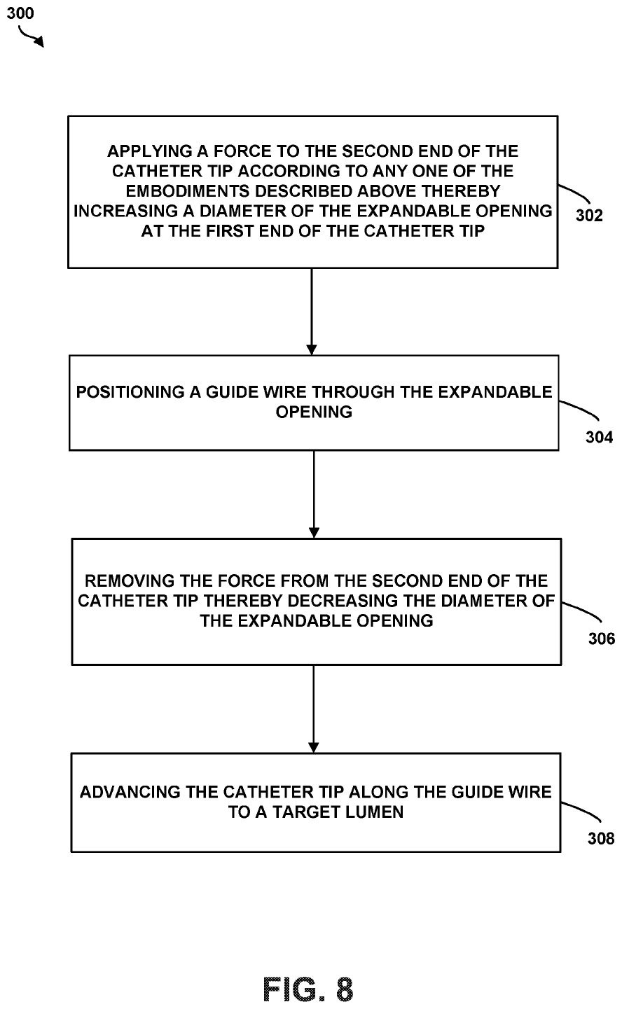

FIG. 8 is a simplified flow chart illustrating another method 300 according to an exemplary embodiment. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In operation, a user may pinch or compress the second end 110 of the catheter tip 100, causing the plurality of pivotable arms 112 to pivot to thereby flex outwardly in a direction away from the cavity 104 thereby increasing the diameter of the expandable opening 106. In practice, the expandable opening 106 of the flexible housing 102 may apply enough pressure on the guide wire to seal the catheter tip 100 against the guide wire to minimize the entry of bodily fluids into the cavity of the catheter tip 100. In addition, the expandable opening 106 may be configured to permit the guide wire to slide back and forth within the catheter tip 100 without binding. In one particular example, the expandable opening 106 may include a lubricating material to reduce any friction between the guide wire and the expandable opening 106.

In particular, at block 302 the method 300 includes applying a force 114 to the second end 110 of the catheter tip 100 according to any one of the embodiments described above thereby increasing a diameter of the expandable opening 106 at the first end 108 of the catheter tip 100. At block 304, the method 300 includes positioning a guide wire through the expandable opening 106. At block 306, the method 300 includes removing the force 114 from the second end 110 of the catheter tip 100 thereby decreasing the diameter of the expandable opening 106. At block 304, the method 300 includes advancing the catheter tip 100 along the guide wire to a target lumen. In another embodiment, the catheter tip 100 is coupled to a catheter 116 configured to have a guide wire-deployable device positioned within a lumen 118 of the catheter. In such an embodiment, the method 300 may further include deploying the guide wire-deployable device from the lumen 118 of the catheter 116 into the target lumen.

In the above description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts were described in conjunction with specific examples, it will be understood that these examples are not intended to be limiting.

1. A catheter tip, comprising:
a flexible housing defining a cavity extending between an expandable opening arranged at a first end of the catheter tip and a second end of the catheter tip; and
a plurality of pivotable arms coupled to and extending longitudinally along the flexible housing, wherein the plurality of pivotable arms are arranged to taper inwardly at the first end of the catheter tip toward a longitudinal axis of the flexible housing, wherein the expandable opening is configured to increase in diameter from a first position to a second position in response to application of an external force to the second end of the flexible housing.

2. The catheter tip of claim 1, wherein the catheter tip has a tapered transition from the first end to the second end, and wherein the cavity at the second end has a greater diameter than at the first end.

3. The catheter tip of claim 1, wherein the expandable opening has a diameter in the first position ranging from about 0.2032 mm to about 0.3556 mm.

4. The catheter tip of claim 1, wherein the expandable opening has a diameter in the second position ranging from about 0.2286 mm to about 1.016 mm.

5. The catheter tip of claim 1, further comprising a catheter coupled to the second end of the catheter tip.

6. The catheter tip of claim 5, further comprising a guide wire-deployable device positioned within a lumen of the catheter, wherein the guide wire-deployable device includes one of an occluder, a pacemaker lead, a snare or a stent graft.

7. The catheter tip of claim 1, wherein the plurality of pivotable arms are embedded in the flexible housing, coupled to an interior of the flexible housing or coupled to an exterior of the flexible housing.

8. The catheter tip of claim 1, wherein the flexible housing is heat shrunk over the plurality of pivotable arms.

9. The catheter tip of claim 1, further comprising:
at least one frame arranged between the plurality of pivotable arms and the cavity and further arranged between a midpoint of each of the plurality of arms and the second end of the catheter tip, wherein the at least one frame is configured as a pivot point for one or more of the plurality of pivotable arms.

10. The catheter tip of claim 9, wherein the at least one frame comprises a ring, a plurality of segments arranged to form a discontinuous ring, a plurality of ball bearings, and/or a plurality of shafts.

11. The catheter tip of claim 1, wherein each of the plurality of pivotable arms has a stiffness greater than a stiffness of the flexible housing.

12. The catheter tip of claim 1, wherein the expandable opening comprises a single ring or a discontinuous ring defined by a first end of each of the plurality of pivotable arms.

13. The catheter tip of claim 1, wherein the plurality of pivotable arms are configured to transition from a linear configuration in which a longitudinal axis of each of the plurality of pivotable arms are in the same plane to a tubular configuration in which the longitudinal axis of each of the plurality of pivotable arms intersect at a center of the expandable opening to thereby form the cavity.

14. The catheter tip of claim 13, wherein a first pivotable arm of the plurality of pivotable arms includes a locking component, and wherein a second pivotable arm of the plurality of pivotable arms includes a key component configured to receive the locking component of the first pivotable arm to thereby form the tubular configuration.

15. The catheter tip of claim 13, wherein each of the plurality of pivotable arms includes a locking component and a key component, and wherein the key component of each of the plurality of pivotable arms is configured to receive the locking component of an adjacent pivotable arm of each of the plurality of pivotable arms to thereby form the tubular configuration.

16. The catheter tip of claim 13, wherein each of the plurality of pivotable arms includes a beveled edge complementary to a beveled edge of an adjacent pivotable arm of each of the plurality of pivotable arms to thereby form the tubular configuration.

17. The catheter tip of claim 1, wherein the expandable opening includes a plurality of ridges or teeth configured to grip a guide wire when the expandable opening is in the first position.

18. The catheter tip of claim 1, wherein the plurality of pivotable arms comprises at least three pivotable arms.

19. The catheter tip of claim 1, wherein each of the plurality of pivotable arms tapers to a point at a first end of the catheter tip.

20. A method of forming a catheter tip, comprising:
forming a plurality of pivotable arms in a linear configuration such that a longitudinal axis of each of the plurality of pivotable arms are in the same plane; and
coupling a first edge of a first pivotable arm of the plurality of pivotable arms to a second edge of a second pivotable arm of the plurality of pivotable arms to form a tubular configuration of the plurality of pivotable arms in which the plurality of pivotable arms define a cavity extending between an expandable opening arranged at a first end of the catheter tip and a second end of the catheter tip.

21.-28. (canceled)
